(19) United States
(12) Patent Application Publication
(10) Pub. No.: US 2019/0029355 A1
(43) Pub. Date: Jan. 31, 2019

(54) HEADBAND FOR MASK

(71) Applicant: SERVORE CO., LTD., Gyeonggi-do (KR)

(72) Inventors: Woon Su SEO, Gyeonggi-do (KR); Jeong Min SEO, Gyeonggi-do (KR)

(21) Appl. No.: 16/000,023

(22) Filed: Jun. 5, 2018

(30) Foreign Application Priority Data
Jul. 27, 2017 (KR) 10-2017-0095186

(51) Int. Cl. A42B 3/14; A42B 3/22

(52) U.S. Cl. CPC A42B 3/145; A61F 9/06; A42B 3/225; A42B 3/142

(57) ABSTRACT

A headband for a mask capable of adjusting a position of a guard, connected to a side of a guard disposed at a position corresponding to the face of a worker and is worn on a side of the worker's head, the headband including: a band worn on a side of the worker's head, having a protrusion partially cut on a side and protruding outward, and having a plurality of grooves continuously formed at a side of the protrusion; a moving body coupled to a side corresponding to a side of the band where the protrusion is formed to be movable in the longitudinal direction of the band; and a rotary lever rotatably disposed at a side of the moving body and rotating such that another side is inserted into or separated out of any one of the plurality of grooves. ∧-shaped inclined portion is formed between the grooves of the protrusion.

### CROSS REFERENCE

The present application claims priority to Korean Patent Application No. 10-2017-0095186, filed 27 Jul. 2017, the entire contents of which is incorporated herein for all purposes by this reference.

### BACKGROUND

The present invention relates to a headband for a mask, and more particularly, to a headband for a mask that allows for easy adjustment of the distance between a guard and the face of a worker and allows for easy adjusting and fixing the position of the guard because a side of a rotary lever can be easily inserted into a groove through an inclined portion at any position when the adjusted position of the guard needs to be fixed.

A welding is a metal process that joins two or more solid metals together. Generally, the welding requires a high energy source. A high pressure electricity or a high pressure and explosive gas such as oxygen, acetylene, or argon etc. are used as the high energy source. The maleficence and hazards according to the welding are caused by welding fumes (metal components contained in the fumes), harmful rays, noises, and high temperature environments etc. that are generated in welding work.

Here, the welding fume is a fine particle generated by a cooling of a material evaporated by a heat during welding and is generated through a diffusion of molten metal vapors due to high-temperature arc heat. In the shielded metal arc welding, the relationship between the amount of fume generation and the welding currents shows a tendency in that the amount of generation increases as a current, a voltage, and a diameter of welding rod increase.

In addition, strong ultraviolet rays and infrared rays, which are harmful rays generated, are fatal to human eyes. Therefore, during welding thereof, welding helmets with shading plates are used to protect workers from the harmful rays and the welding fumes.

FIG. 1A is a perspective view showing a conventional welding mask and FIG. 1B is a perspective view showing a distance control unit according to the conventional welding mask.

As shown in FIG. 1A and FIG. 1B, the conventional welding mask includes a mask body (110) disposed in front of a face of a worker and having a light-shading plate (111) formed on one side thereof corresponding to an eye of the worker, a headband (120) connected to a rear side of the main body (110) and having one side supported on the head of the worker, and a distance control unit (130) coupled to one side of the headband (120).

In the conventional welding mask, the headband (120) is worn to cover the head of the worker, so that the mask body (110) is disposed at a position corresponding to the face of the worker. During working thereof, the light-shading plate (111) detects light and automatically shades it so as to perform the working thereof.

In this case, when the conventional welding mask (100) is used commonly by a plurality of workers, the distance control unit (130) is adjusted, so that each worker appropriately adjusts the position of the mask spaced from the face according to the head size and the face shape thereof.

More concretely, the distance control unit (130) according to the conventional welding mask includes a protrusion (131) protruding outward by partially cutting out one side of the headband (120), a plurality of distance control grooves (131a) formed on one side of the protrusion (131) at regular intervals, a slider (132) movably installed on one side of the headband (120) and having one side coupled to the mask body (110), and a coupling protrusion (132a) formed on one side of the slider (132) and inserted into any one of the distance control grooves (131a). The mask body (110) is moved back and forth through the slider 132, so that the position of the mask body (110), which is adjusted through the coupling protrusion (132a) formed at one side of the slider (132), is fixed.

However, in the conventional welding mask (100), in order to insert the coupling protrusion (132a) into the distance control grooves (131a) formed at a predetermined position, since it is necessary to insert the coupling protrusion (132a) into the corresponding distance control groove (131a) in a state that the position of coupling protrusion (132a) and the position of any one of the distance control grooves (131a) are accurately matched with each other, the operation for accurately positioning the coupling protrusion (132a) and the distance control groove (131a) should be preceded so as to fix the mask body 110. Also, where the worker is unable to find the correct position of the distance control groove (131a) in a state that the welding mask (100) is worn by the worker, since it is necessary to remove the welding mask (100) again and insert the coupling protrusion (132a) into the distance control groove (131a), there is a problem in that the operation thereof is very troublesome.

### SUMMARY OF THE INVENTION

The present invention has been made in consideration of the problems described above and an object of the present invention is to provide a headband for a mask that allows for easy adjustment of the distance between a guard and the face of a worker and allows for easy adjusting and fixing the position of the guard because a side of a rotary lever can be easily inserted into a groove through an inclined portion at any position when the adjusted position of the guard needs to be fixed.

According to an aspect of the invention to achieve the object described above, there is provided a headband for a mask that is connected to a side of a guard disposed at a position corresponding to the face of a worker and is worn on a side of the worker's head, the headband including: a band worn on a side of the worker's head, having a protrusion partially cut on a side and protruding outward, and having a plurality of grooves continuously formed at a side of the protrusion; a moving body coupled to a side corresponding to a side of the band where the protrusion is formed to be movable in the longitudinal direction of the band; and a rotary lever rotatably disposed at a side of the moving body and rotating such that another side is inserted into or separated out of any one of the plurality of grooves.

Preferably, ∧-shaped or ∩-shaped inclined portions are formed between the grooves of the protrusion.

Preferably, the moving body includes a moving member coupled to a side of the band where a prominence-depression portion is formed to be movable in the longitudinal direction of the band; and a guard coupler extending outward from a side of the moving member and coupled to a side of the guard coupler at a side.

Preferably, the guard coupler includes an extending plate extending downward from a side of the moving member; a coupling plate rotatably coupled to a side of the extending plate and having a coupling protrusion formed on a side to be inserted in a coupling hole formed at a side of the guard; and a pressing portion coupled to a side of the coupling plate with a side of the guard therebetween and pressing and fixing a side of the guard.

Preferably, the rotary lever includes a rotary body rotatably coupled to a side of the moving body; a position fixer formed in a shape corresponding to the fixing groove on a side of the rotary body, and inserted into or separated out of the fixing groove with rotation of the rotary body; and a holding portion extending outward from a side of the rotary body.

Preferably, the headband has a plurality of fixing holes formed with intervals corresponding to the intervals of the plurality of grooves at a side, and the rotary lever further includes a locking portion extending from a side of the rotary body such that a side is positioned to face the rotary body with the band therebetween and having a fixing protrusion protruding toward the band from a side such that when the position fixer is inserted into the fixing groove, the fixing protrusion is inserted into the fixing hole.

### BRIEF DESCRIPTION OF THE DRAWINGS

The above and other objects, features and advantages of the present invention will be more apparent from the following detailed description taken in conconfluence with the accompanying drawings, in which:

FIG. 1A is a perspective view showing a conventional welding mask;

FIG. 1B is a perspective view showing a distance control unit according to the conventional welding mask;

FIG. 2 is a perspective view of a headband for a mask according to an embodiment of the present invention;

FIG. 3 is an exploded perspective view of the headband for a mask according to an embodiment of the present invention;

FIG. 4 is a partial cross-sectional view of the headband for a mask according to an embodiment of the present invention;

FIG. 5 is a view showing a state in which the welding face according to an embodiment of the present invention is moved in the front-rear direction;

FIGS. 6A and 6B are views showing a state in which the position fixer according to an embodiment of the present invention is inserted into a groove; and

FIG. 7 is a view showing a state in which the angle of the welding face according to an embodiment of the present invention is adjusted.

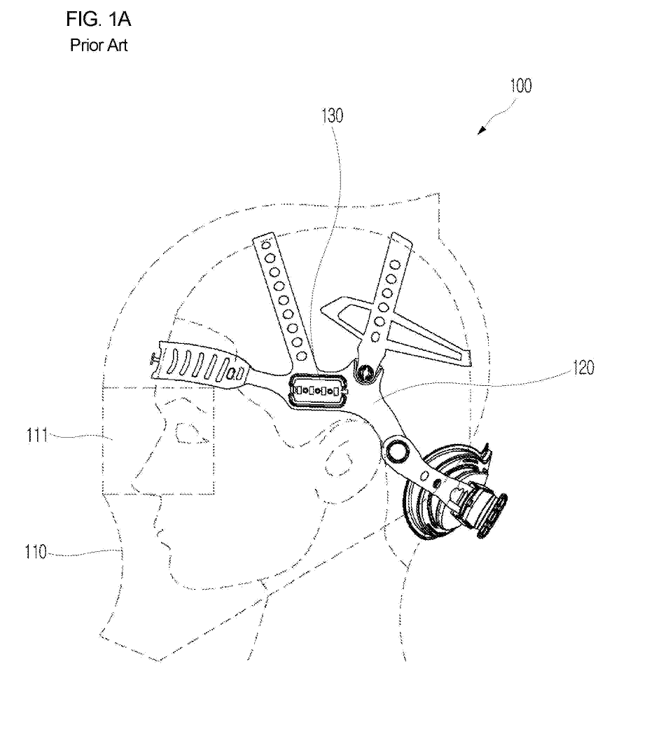

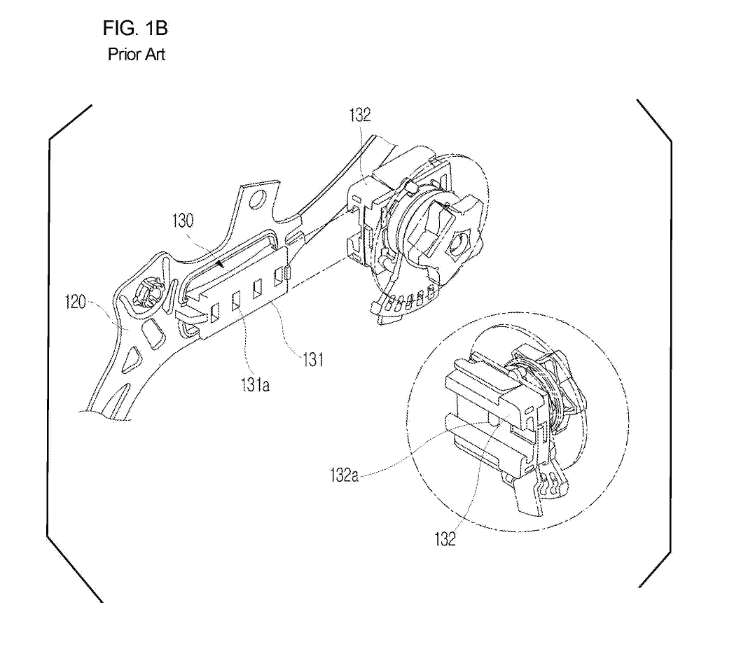

### DETAILED DESCRIPTION OF THE PREFERRED EMBODIMENT

Hereinafter, a preferred embodiment according to the present invention will be described in detail with reference to the accompanying drawings. Since the description of the present invention is a mere embodiment for structural and functional description, it must not be interpreted that the scope of the present invention is limited by the embodiments described in the text. That is, since the embodiments can be variously changed and have various forms, it should be understood that the scope of the invention includes the equivalents for realizing the technical concept. Also, since the specific embodiments do not include all objects and effects presented by the present invention, the scope of the present invention is not limited by them. This invention can be implemented in many different forms without departing from technical aspects or main features.

Hereinafter, a preferred embodiment according to the present invention will be described in detail with reference to the accompanying drawings.

A headband for a mask of the present invention can be applied to all product having a guard for protecting the face or breath of a worker by being in front of the worker's face such as a welding mask, a safety helmet, and a breathing mask and it is exemplified in the embodiment that the headband of the present invention is applied to a headband for welding masks for the convenience of description.

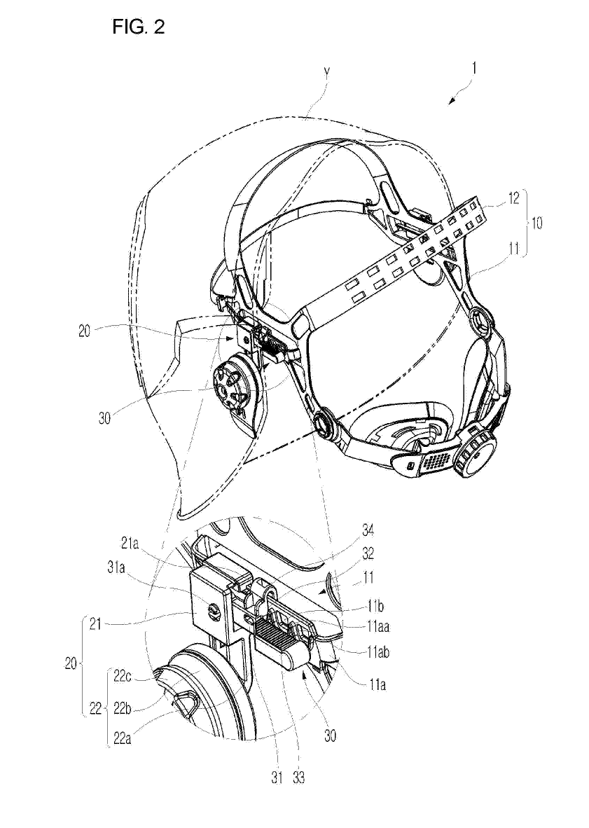

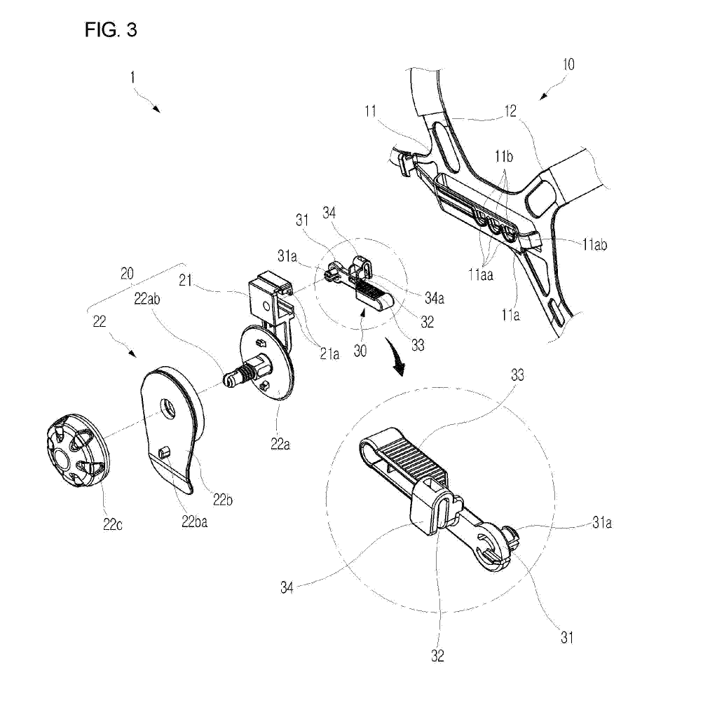

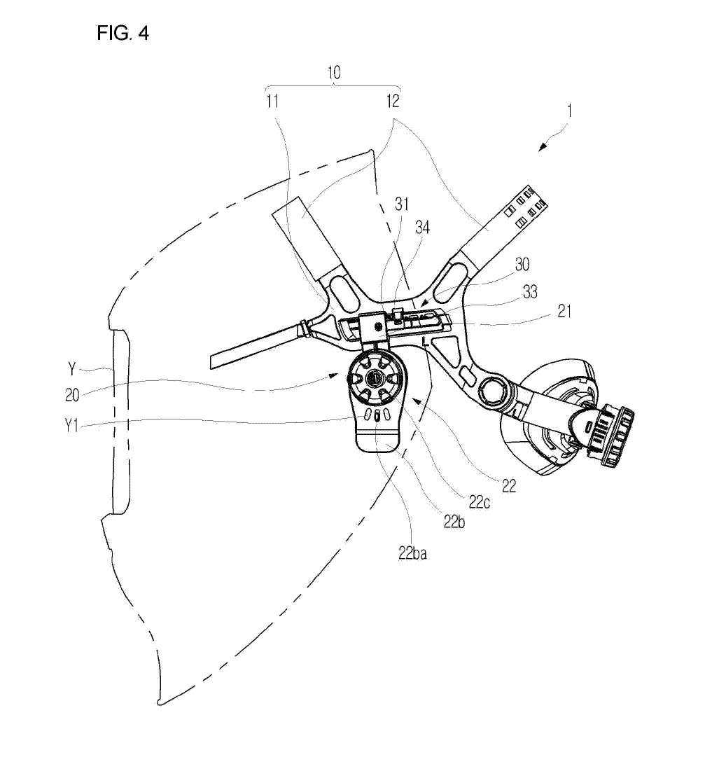

FIG. 2 is a perspective view of a headband for a mask according to an embodiment of the present invention, FIG. 3 is an exploded perspective view of the headband for a mask according to an embodiment of the present invention, and FIG. 4 is a partial cross-sectional view of the headband for a mask according to an embodiment of the present invention.

As shown in FIGS. 2 to 4, the headband for a mask according to an embodiment of the present invention includes a band (10), a moving body (20), and a rotary lever (30).

The band (10) is formed substantially in a belt shape, is combined with a welding face (Y) at a side through the moving body (20) to be described below, and allows the guard of a welding mask, that is, the welding face (Y) of a welding mask to be positioned to correspond to the face of a worker when worn on a side of the worker's head.

The band (10) may include a first band unit (11) that is put around the head of a worker so that the band can be more stably worn on the worker's head and a second band unit (12) having both ends coupled to a side of the first band unit (11) and surrounding the upper portion of the worker's head. A latch unit allowing for adjustment of the length of the first band unit (11) may be disposed at the center of the rear side of the first band unit (11).

In the band (10), protrusions (11a) protruding outward are formed at both sides of the first band unit (11), a plurality of grooves (11aa) is continuously formed at the upper portions of the protrusions (11a), and ∧-shaped or ∩-shaped inclined portions (11ab) are formed between the grooves (11aa).

The ∧-shaped or ∩-shaped inclined portions (11ab) allow the welding face (Y) to be smoothly fixed by naturally coming into the grooves (11a) along the inclined portions (11ab) even though position fixers (32) of rotary levers (30) to be described below are not accurately fit into the plurality of grooves (11aa) when a worker fixes the welding face (Y) of the working mask by turning the rotary levers (30) at a desired position.

Further, a plurality of fixing holes (11b) are formed at a side of the first band (11) with intervals corresponding to the intervals of the plurality of grooves (11aa).

The moving body (20) includes a moving member (21) supported at a side of the band (10) having a prominence-depression portion and coupled to be movable in the longitudinal direction of the band (10) and a guard coupler (22) extending downward from a side of the moving member (21). The moving body (20) is moved forward/backward in the longitudinal direction of the band (10) with both ends of the welding face (Y) coupled to a side thereof such that the position of the welding face (Y) can be adjusted from the face of a worker.

The moving member (21) formed in a substantially ⊃-shaped block with the upper and lower inner sides supported by upper and lower sides of the band (10) and has a stopper (21a) extending inward at both ends to be supported on the inner sides of the band (10). The moving member (21) is moved in the longitudinal direction of the band (10) such that the welding face (Y) coupled to the guard coupler (22) and the rotary lever (30) coupled to a side are also moved in the longitudinal direction of the band (10).

The guard coupler (22) includes an extending plate (22a) extending downward from a side of the moving member (21), a coupling plate (22b) rotatably coupled to a side of the extending plate (22a), and a pressing portion (22c) coupled to a side of the coupling plate (22b) with a side of the welding face (Y) therebetween. When the moving member (21) supporting a side of the welding face (Y) is moved in the longitudinal direction of the band (10), the welding face (Y) is also moved.

The extending plate (22a) is formed in a substantially plate shape, provides an area for installing the coupling plate (22b) and the pressing portion (22c), and supports the coupling plate (22b) and the pressing portion (22c).

The coupling plate (22b) has a threaded protrusion (22aa) protruding outward from the outer side through a side of the welding face (Y).

The coupling plate (22b) is formed substantially in a disc shape, is rotatably coupled to a side of the extending plate (22a), as described above, has a coupling protrusion (22ba) protruding outward from the outer side, and is inserted in a coupling hole (Y1) formed at an end of the welding face (Y), thereby being combined with the welding face (Y).

A plurality of coupling holes (Y1) is formed at each of both ends of the welding face (Y), so the wearing angle of the welding face (Y) can be adjusted by selectively inserting the coupling protrusions (22ba) to which both ends of the welding face (Y) correspond into any one of the plurality of coupling holes (Y1).

The pressing portion (22c) is formed substantially in a circular cap shape, is thread-fastened to the threaded protrusion (22aa) of the coupling plate (22b) with a side of the welding face (Y) therebetween, and more firmly supports the welding face (Y) by pressing the outer side of the welding face (Y).

The rotary lever (30) includes a rotary body (31) rotatably coupled to a side of the moving body (20), a position fixer (32) formed on a side of the rotary body (31) to be inserted into or separated out of the grooves (11aa), a holding portion (33) extending outward from a side of the rotary body (31), and a locking portion (34) locked to a side of the band (10) by being inserted into a fixing hole (11b) at a side when the position fixer (32) is inserted into the grooves (11aa). The rotary lever (30) fixes the position of the welding face (Y) adjusted by the moving body (20).

The rotary body (31) is formed substantially in a block shape, has a coupling pin (31a) on a side, is rotatably coupled to a side of the moving member (21) through the coupling pin (31a), and rotates such that the position fixer (32) is inserted into or separated out of the grooves (11aa).

The position fixer (32) protrudes from a side of the rotary body (31) and is formed in a shape corresponding to the grooves (11aa), so when the rotary body (31) is rotated, the position fixer (32) fixes the position of the welding face (Y) by being inserted into the grooves (11aa).

The lower end of the position fixer (32) is curved, so when the position fixer (32) is rotated toward a fixing groove by the rotary body (31) between the grooves (11aa), the curved portion is more easily guided and inserted into the grooves (11aa) by the inclined portions (11ab) formed between the grooves (11aa).

The holding portion (33), as described above, extends outward from a side of the rotary body (31) to provide a holding area for a worker. The holding portion (33) provides the holding area for rotating the rotary body (31), so the rotary body (31) can be more easily rotated.

The locking portion (34) is formed substantially in a ∩-shape from the upper portion of the rotary body (31) such that a side is positioned to face the rotary body (31) with the band (10) therebetween, and has a fixing protrusion protruding toward the band (10) on a side.

When the position fixer (32) is inserted into the grooves (11aa), the fixing protrusion is inserted into any one of the plurality of fixing holes (11b) and locked to a side of the band (10), so the locking portion (34) firmly fixes the position of the position fixer (32) inserted in the fixing groove, that is, the adjusted position of the welding face (Y).

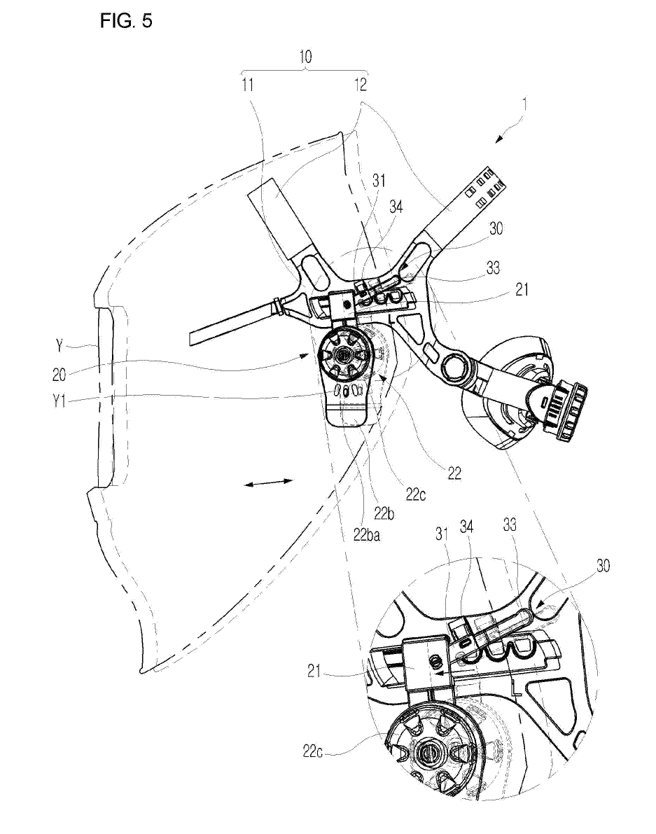

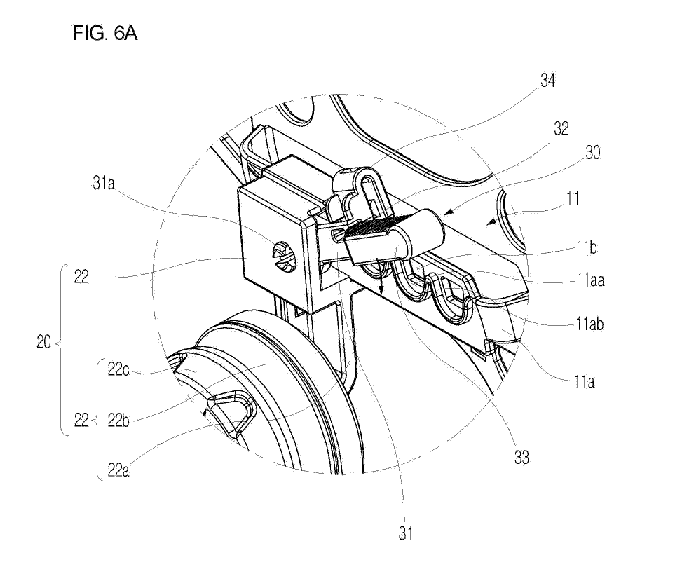

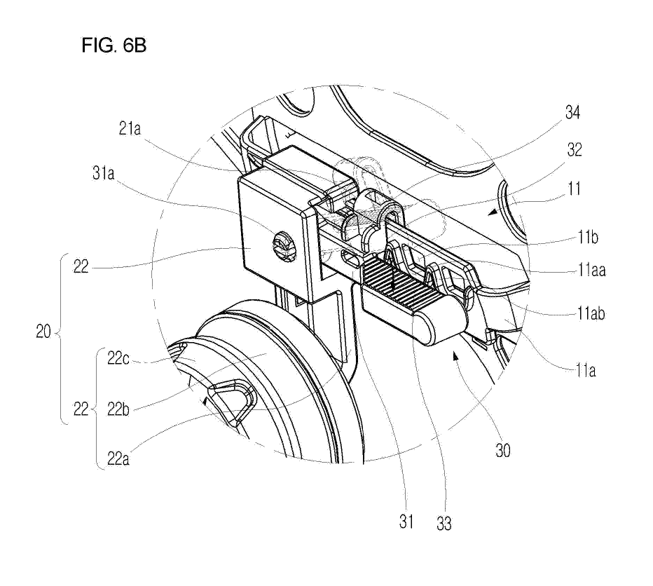

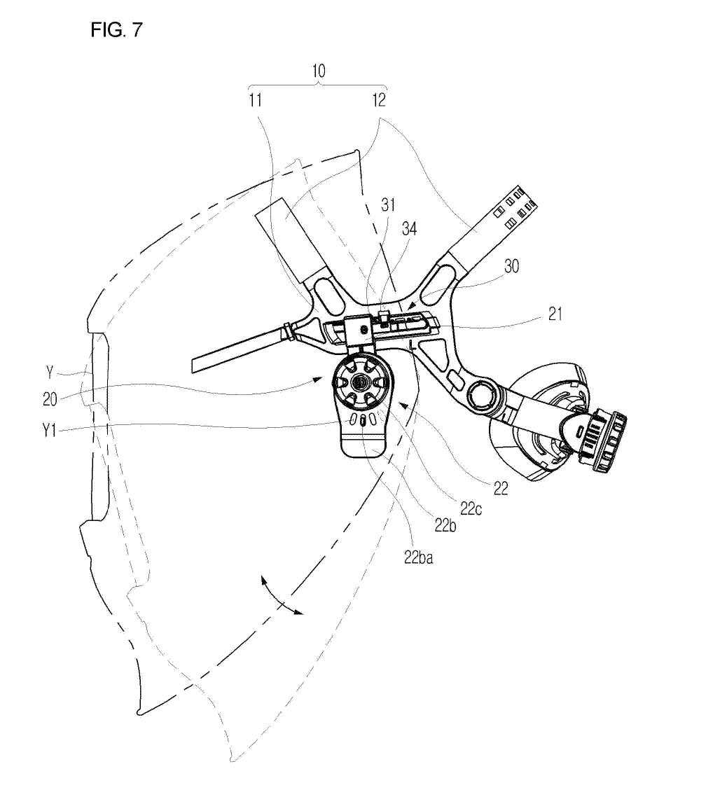

FIG. 5 is a view showing a state in which the welding face according to an embodiment of the present invention is moved in the front-rear direction, FIGS. 6A and 6B are views showing a state in which the position fixer according to an embodiment of the present invention is inserted into a groove, and FIG. 7 is a view showing a state in which the angle of the welding face according to an embodiment of the present invention is adjusted.

The operation of the headband for a mask having the configuration according to an embodiment of the present invention is described hereafter with reference to FIGS. 5 to 7.

First, a worker puts the headband (H) of a welding mask on his/her head with the welding face (Y) of the welding mask positioned to correspond to his/her face.

Next, the worker holds the holding portion (33) with the position fixer (32) of the rotary lever (30) separated out of the fixing groove and appropriately adjusts the position of the welding face (Y) from his/her face by moving the moving member (21) in the longitudinal direction of the band (10).

When finishing adjusting the position of the welding face (Y), the worker holds the holding portion (33) and rotates down the rotary body (31) such that the position fixer (32) formed on a side of the rotary body (31) is inserted into any one of the plurality of grooves (11aa).

When the position fixer (32) is positioned between the grooves (11aa), the curved lower end is guided to a groove (11aa) along the inclined portion (11ab) formed between the grooves (11aa), so it is easily inserted into the groove (11aa). Further, as the position fixer (32) is inserted into the groove (11aa), the adjusted position of the welding face (Y) is fixed.

Thereafter, when the worker wants to adjust the angle of the welding face (Y), as shown in FIG. 7, he/she releases the pressing state of the welding face (Y) by rotating the pressing portion (22c), holds and opens both ends of the welding face (Y), selectively inserts the coupling protrusion (22ba) into any one of the plurality of coupling holes (Y1) formed at the welding face (Y), and then rotates the pressing portion (22c) backward to press a side of the welding face (Y), whereby the angle of the welding face (Y) is adjusted.

According to the present invention, there are effects in that it allows for easy adjustment of the distance between a guard and the face of a worker and allows for easy adjusting and fixing the position of the guard because a side of a rotary lever can be easily inserted into a groove through an inclined portion at any position when the adjusted position of the guard needs to be fixed.

While the present invention has been described with respect to the specific embodiments, it will be apparent to those skilled in the art that various changes and modifications may be made without departing from the spirit and scope of the invention as defined in the following claims.

What is claimed is:

1. A headband for a mask that is connected to a side of a guard disposed at a position corresponding to the face of a worker and is worn on a side of the worker's head, the headband comprising:
a band worn on a side of the worker's head, having a protrusion partially cut on a side and protruding outward, and having a plurality of grooves continuously formed at a side of the protrusion;
a moving body coupled to a side corresponding to a side of the band where the protrusion is formed to be movable in the longitudinal direction of the band; and
a rotary lever rotatably disposed at a side of the moving body and rotating such that another side is inserted into or separated out of any one of the plurality of grooves.

2. The headband according to claim 1, wherein ∧-shaped or ∩-shaped inclined portions are formed between the grooves of the protrusion.

3. The headband according to claim 1, wherein the moving body includes:
a moving member coupled to a side of the band where a prominence-depression portion is formed to be movable in the longitudinal direction of the band; and
a guard coupler extending outward from a side of the moving member and coupled to a side of the guard coupler at a side.

4. The headband according to claim 3, wherein the guard coupler includes:
an extending plate extending downward from a side of the moving member;
a coupling plate rotatably coupled to a side of the extending plate and having a coupling protrusion formed on a side to be inserted in a coupling hole formed at a side of the guard; and
a pressing portion coupled to a side of the coupling plate with a side of the guard therebetween and pressing and fixing a side of the guard.

5. The headband according to claim 1, wherein the rotary lever includes:
a rotary body rotatably coupled to a side of the moving body;
a position fixer formed in a shape corresponding to the fixing groove on a side of the rotary body, and inserted into or separated out of the fixing groove with rotation of the rotary body; and
a holding portion extending outward from a side of the rotary body.

6. The headband according to claim 5, wherein the headband has a plurality of fixing holes formed with intervals corresponding to the intervals of the plurality of grooves at a side, and
the rotary lever further includes a locking portion extending from a side of the rotary body such that a side is positioned to face the rotary body with the band therebetween and having a fixing protrusion protruding toward the band from a side such that when the position fixer is inserted into the fixing groove, the fixing protrusion is inserted into the fixing hole.
